(19) United States
(12) Patent Application Publication
(10) Pub. No.: US 2021/0121645 A1
(43) Pub. Date: Apr. 29, 2021

(54) SINGLE-USE DISPENSER WITH MULTIPLE DELIVERY OPTIONS

(71) Applicant: NS3 Biotech LLC, Dover, DE (US)

(72) Inventors: Christopher Ginocchetti, St Pete Beach, FL (US); Fady Boctor, Chester Springs, PA (US)

(21) Appl. No.: 16/665,153

(22) Filed: Oct. 28, 2019

(51) Int. Cl. A61M 15/00; B65D 83/00; B65D 83/42

(52) U.S. Cl. CPC A61M 15/009; A61K 9/0043; B65D 83/42; B65D 83/0005

(57) ABSTRACT

A dispenser including a reservoir configured for holding a fluid and more than one nozzle fluidly connected to the reservoir, wherein one of the more than one nozzle is a nozzle configured for dispensing one or more drops of the fluid and another one of the more than one nozzle is a nozzle configured for dispensing a spray, wherein the reservoir is configured to be squeezable to force the fluid to at least one of the more than one nozzle to be dispensed.

### BACKGROUND OF THE INVENTION

### 1. The Field of the Invention

The present invention relates to a single-use dispenser for one or more orifices of the human body. More specifically, the present invention is directed to a single-use dispenser for one of a plurality of applicable orifices of the human body where the selection of the appropriate nozzle for use on the selected orifice is made at the time of use.

### 2. Background Art

Single-use dispensers of a liquid, e.g., saline, etc., configured for dispensing drops have been attempted in the health care industry. A single-use dispenser is a device having a reservoir holding a sufficient amount of fluid for a one-time use where the fluid is to be dispensed through a nozzle that is meant to be unblocked or exposed only just prior to its use. Any remaining and unused amount is to be discarded as the fluid contained therein is not preserved upon its exposure to the ambient environment. Prior art single- use dispensers are configured for dispensing only drops instead of a spray, e.g., a fine mist or spray. Therefore, prior art single-use dispensers are unsuitable for applications where more energetic streams of fluid are required, e.g., to dislodge mucous and other bodily fluids and solids from the nasal passage and sinuses and to allow a greater reach of such fluid within these parts of one's body. Users that require energetic streams must resort to the use of multi-use dispensers which often times contain preservatives as their contents must remain effective over a long period of time. Further, a user who desires to have access to the various modes of dispensing at moments notice will find it difficult to maintain multiple dispensers of significant sizes all of the time. Therefore, such a user will need to be concerned with replenishing his or her stock of solutions, e.g., saline, often to make sure that when saline and other solutions are required, they are readily available in the desired format. Yet further, significant amounts of fluids may be discarded and wasted as they have remained unused after the expiration dates that mark the loss of their efficacy as they have been packaged in large containers or dispensers. Even if there had been a single-use dispenser adapted for dispensing a fine mist or spray, the need for discretely-available dispensers for dispensing drops and fine mist or spray would require different types of dispensers to be made available, reducing the chance that a particular type of single-use dispenser would not be available when it is required. The ensuing patent references disclose dispensers of eye drops.

Korean Pat. Pub. No. 10-1927756 discloses an eye drop container having a body with an eye drop storage space formed therein, an eye drop discharging unit extending from the container body to form an eye drop moving path connected to the eye drop discharging port, and a sealing unit connected to the eye drop discharging unit, thereby discharging the eye drops in the eye drop storing space. This publication is silent regarding a container having more than one nozzle selectable for dispensing the contents of the container although it demonstrates a way in which the container can be deformed to dispense the contents.

Chinese Pat. Pub. No. 204246502 discloses a low-density polyethylene portable eye drop bottle for storing and dispensing a single dosage of medicine. Again, this publication is silent regarding a container having more than one nozzle selectable for dispensing the contents of the container.

U.S. Pat. No. 6,241,124 to Hoyt (Hereinafter Hoyt) discloses a single-use container useful for the storage and dispensation of small quantities of liquids. The subject container is particularly useful for dispensing sterile, preservative-free formulations, such as those used in single dose eye drop applications. Hoyt is silent regarding a container having more than one nozzle selectable for dispensing the contents of the container although there is disclosed a manner in which the container can be squeezed such that its contents can be dispensed.

U.S. Pat. Pub. No. 20140323973 of Ruiz (Hereinafter Ruiz) discloses a pure sodium chloride is dissolved in sterile distilled water to form a sterile pure saline solution. A hermetically sealed disposable spray container holds a single measured dosage of the solution for fully rinsing and cleansing a nasal passageway. The measured dosage is sufficient for rinsing and cleansing the nasal passage and harmless if the measured dosage mistakenly enters the lungs or sinuses or stomach of the user. Ruiz is silent regarding a container having more than one nozzle selectable for dispensing the contents of the container although a manner in which the container can be squeezed to empty its contents is disclosed.

U.S. Pat. No. 3,356,244 to Witchell (Hereinafter Witchell) discloses a container including a one-piece container body and cap and a neck of reduced diameter between the body and cap. The cap is to be removed by applying torsional stress to the neck portion, whereby it will shear. Witchell is silent regarding a container having more than one nozzle selectable for dispensing the contents of the container although it demonstrates a way to twist a cap such that the contents of the container can be accessed.

None of the references disclosed herein are geared towards minimizing wastes by combining two dispensing options in a single dispenser. There exists a need for a single-use dispenser with multiple delivery options such that a user need only carry one type of dispensers for use on various orifices of a human body.

### SUMMARY OF THE INVENTION

In accordance with the present invention, there is provided a dispenser including:

(a) a reservoir configured for holding a fluid; and

(b) more than one nozzle fluidly connected to the reservoir, wherein one of the more than one nozzle is a nozzle configured for dispensing one or more drops of the fluid and another one of the more than one nozzle is a nozzle configured for dispensing a spray, wherein the reservoir is configured to be squeezable to force the fluid to at least one of the more than one nozzle to be dispensed.

In one embodiment, the dispenser further includes a neck disposed on one of the more than one nozzle and a shoulder at a base of the neck, the neck is configured to extend the reach of the more than one nozzle into a nostril and the shoulder is configured to limit insertion of the one of the more than one nozzle to prevent injury from being imparted on the nostril.

In one embodiment, at least a portion of the neck includes a weak wall configured to be breakable from a twist of the cap against the neck such that the fluid can be dispensed through the one of the more than one nozzle.

In one embodiment, the neck is disposed at a first size at the cap and a second size away from the cap, the second size is greater than the first size and the neck is configured to taper from the first size to the second size at a taper such that the taper causes the one of the more than one nozzle to be centered while the one of the more than one nozzle is seated against the nostril.

In one embodiment, the more than one dispenser includes a drop dispenser. In one embodiment, the more than one dispenser includes a spray dispenser.

In one embodiment, the dispenser includes a breakable member having two sides, one of the two sides is configured to be connected to the dispenser and the other one of the two sides is connected to another one of such dispensers. In one embodiment, the one of the more than one nozzle includes a screen to cause the fluid to exit the one of the more than one nozzle in a fine mist or spray form.

In one embodiment, the more than one nozzle includes two nozzles.

An object of the present invention is to provide a multipurpose dispenser capable of dispensing in more than one mode, a liquid contained in a single-use container.

Another object of the present invention is to provide a multipurpose dispenser capable of dispensing in a drop or fine mist or spray form, a liquid contained in a single-use container.

Another object of the present invention is to provide a multipurpose dispenser capable of dispensing in a drop or fine mist or spray form, a liquid contained in a hermetically- sealed container.

Whereas there may be many embodiments of the present invention, each embodiment may meet one or more of the foregoing recited objects in any combination. It is not intended that each embodiment will necessarily meet each objective. Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification.

### BRIEF DESCRIPTION OF THE DRAWINGS

In order that the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a top front perspective view of one embodiment of the present dispenser.

FIG. 2 is a top view thereof.

FIG. 3 is a bottom view thereof.

FIG. 4 is a front view thereof.

FIG. 5 is a rear view thereof.

FIG. 6 is a right side view thereof.

FIG. 7 is a front view of the embodiment shown in FIG. 1 with the cap of the nasal nozzle removed.

FIG. 8 is a rear view of the embodiment shown in FIG. 1 with the cap of the ear or eye nozzle removed.

FIG. 9 is a diagram depicting a manner in which the nasal nozzle can be used.

FIG. 10 is a diagram depicting a manner in which the dispenser for ear or eye can be used on a user's eye.

FIG. 11 is a diagram depicting a manner in which the dispenser for ear or eye can be used on a user's ear.

FIG. 12 is a top view of a plurality of dispensers supplied as a single group of dispensers.

FIG. 13 is a top front perspective view thereof.

FIG. 14 is a top rear perspective view thereof.

### PARTS LIST

2—dispenser

4—reservoir

6—cap for nasal nozzle

8—neck of nasal nozzle

10—cap for ear or eye nozzle

12—neck of ear or eye nozzle

14—indicia indicating nasal nozzle

16—indicia indicating ear or eye nozzle

18—width of cap for nasal nozzle

20—width of base of neck of nasal nozzle

22—width of cap for ear or eye nozzle

24—width of base of neck of ear or eye nozzle

26—length of cap for nasal nozzle

28—length of cap for ear or eye nozzle

30—length of neck of nasal nozzle

32—length of neck of ear or eye nozzle

34—length of midsection

36—height of dispenser

38—screen

40—slit

42—joint

44—user's hand

46—user's eye

48—user's nostril

50—drop

52—fine mist

54—user's thumb

56—user's index finger

58—user's nose

60—cut

62—ear

### PARTICULAR ADVANTAGES OF THE INVENTION

The present dispenser readily provides a user with a choice of a drop or a spray nozzle all in a single dispenser. Therefore, a user does not need to be concerned with whether the dispenser is used for the nose, ear or eye as long as he or she chooses the right nozzle of the dispenser and the liquid formulation is studied or labeled safe for multi- orifice delivery. Further, if the present single-use dispensers can be used for multiple purposes, the likelihood that a dispenser exceeds its shelf life while in storage is lowered, potentially reducing wastes due to expiration of the contents of these dispensers. In the present dispenser, a reservoir of fluid is equipped with two nozzles, a first of the two nozzles is configured to dispense the liquid in a drop format and a second of the two nozzles is configured for dispensing the liquid in a fine or variable mist format. Depending on the intended usage, a user of the dispenser may choose to use the first nozzle or the second nozzle or both nozzles. However upon the use of at least one of the nozzles, the dispenser is considered used and may not be saved for future use due to potential contamination and exposure of the contents of the liquid to the ambient environment which causes a reduction in its effectiveness and/or sterility. The waste of significant amounts of fluids is reduced as only a small amount of fluid, if any, is wasted as each single-use dispenser is only allocated a small amount suitable for only a single use.

The present dispenser includes two ends, each equipped with a nozzle. At one end, the nozzle is configured for the nose and the other end, for the ear or eye. The nozzles are each fashioned with a unique neck. A user may select the right nozzle to use even without direct visual confirmation of the type of nozzle required as the user can select by feel or touch due to the unique necks, e.g., one of the nozzles includes a neck that is significantly longer than the neck of the other nozzle indicating intranasal use design.

One end of the present dispenser is equipped with a nozzle having a neck capable of being inserted in a user's nostril such that the nozzle is disposed close to its target for effective use of the contents of the dispenser on the user.

### DETAILED DESCRIPTION OF A PREFERRED EMBODIMENT

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

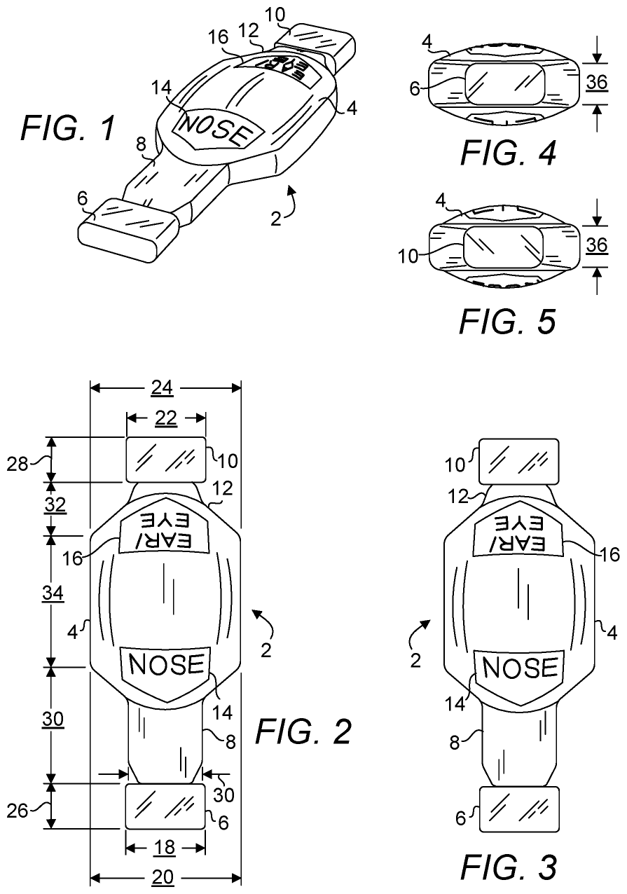

FIG. 1 is a top front perspective view of one embodiment of the present dispenser 2. FIG. 2 is a top view thereof. FIG. 3 is a bottom view thereof. FIG. 4 is a front view thereof. FIG. 5 is a rear view thereof. FIG. 6 is a right side view thereof. The dispenser 2 includes a reservoir 4 for holding a fluid, two nozzles, each connected to the reservoir 4 and each nozzle is sealed with a cap 6, 10 such that the fluid is confined in the reservoir 4, isolated from an ambient environment while in storage. The reservoir 4 is configured to be squeezable to force the fluid to one of the nozzles to be dispensed. As there are two nozzles to choose from, a user can select whether to use one which dispenses drops, e.g., for eyes and ears or the other one which dispenses a fine mist or spray, e.g., in a nostril. The dispenser 2 further includes a neck disposed on each of the nozzles, i.e., the drop nozzle and fine mist or spray nozzle. Neck 12 is configured to extend slightly from the reservoir 4 to facilitate the use of drops on the ear or eye nozzle. Neck 8 is configured to extend the reach of the nasal nozzle in a nostril and at the base of the neck is a shoulder useful for limiting insertion of the nasal nozzle in the nostril to prevent injury from being imparted on the nostril. Upon selecting the desired nozzle, the cap for that nozzle can be removed by the user by grasping the dispenser by the cap and the reservoir 4 or one of the necks 8, 12. Referring back to FIG. 2, in one embodiment, the width 18 of the cap for the nasal nozzle is about 10 mm. The width 20 of the base of the neck of the nasal nozzle is about 25 mm. The width 22 of the cap for the ear or eye nozzle is about 10 mm. The width 24 of the base of the neck of the ear or eye nozzle is about 25 mm. The length 26 of the cap for the nasal nozzle is about 5 mm. The length 28 of the cap for the ear or eye nozzle is about 5 mm. The length 30 of the neck of the nasal nozzle is about 20 mm. The length 32 of the neck of the ear or eye nozzle is about 10 mm. The length 34 of the midsection is about 25 mm. The height 36 of the dispenser is about 5 mm.

On the top face of the dispenser, an indicium is provided to indicate the purpose of each of the nozzles. For instance, an indicium labeled “NOSE” indicates that the nozzle disposed at that end is suitable for dispensing the contents of the reservoir 4 into the nostril of a user. An indicium labeled “EAR/EYE” indicates that the dispenser disposed at that end is suitable for dispensing the contents of the reservoir 4 into an eye or ear of a user. In one embodiment, at least a portion of each neck includes a weak wall configured to be breakable from a twist of the cap against the neck such that the fluid contained in the reservoir can be dispensed through one of the nozzles. In one embodiment, each neck is disposed at a first size at the cap and a second size away from the cap, the second size is greater than the first size and the neck is configured to taper from the first size to the second size at a taper such that the taper causes a nozzle to be centered while the nozzle is seated against the nostril. In one embodiment, one of the nozzles includes a drop dispenser. In one embodiment, one of the nozzles includes a spray dispenser. In one embodiment, the dispenser includes a breakable member having two sides, one of the two sides is configured to be connected to a dispenser and the other one of the two sides is connected to another one of such dispensers.

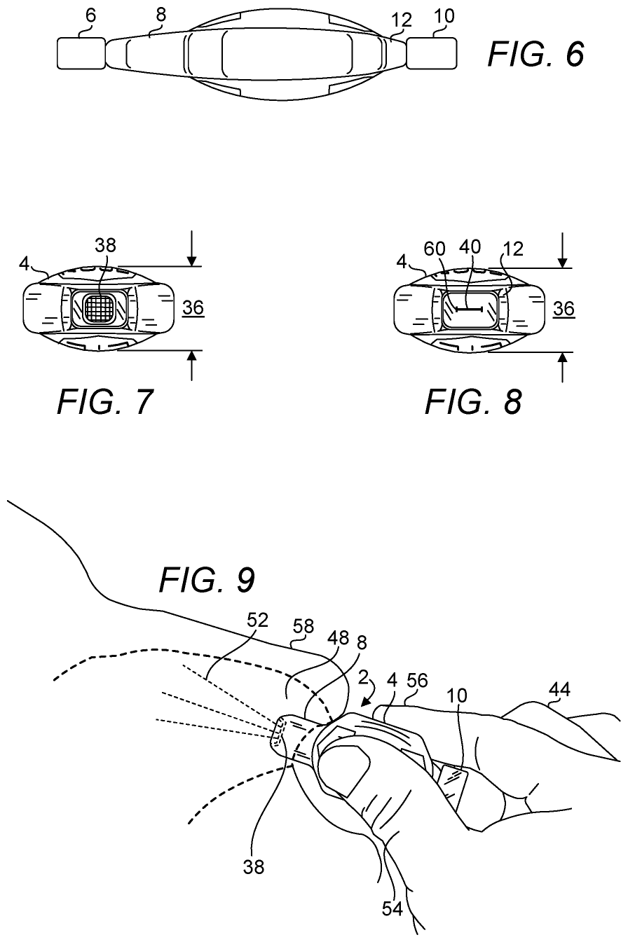

FIG. 7 is a front view of the embodiment shown in FIG. 1 with the cap of the nasal nozzle removed. In one embodiment, the nozzle labelled with indicia “NOSE” 14 includes a screen 38 disposed at or near the tip of neck 8 to cause the fluid to exit the nozzle in a fine mist or spray form. The screen is essentially any barrier with fine apertures or porous barrier which allow penetration of fluids (air and saline solution) when it is subjected to a pressure sufficiently high that is supplied by a squeezing action of the user aided by pressurization in the reservoir 4. Upon removing the cap of the spray nozzle, the apertures of the screen are configured such that the pressurization of the reservoir 4 is not lost, i.e., the contents of the reservoir 4 which include gas and liquid are disposed at a pressure largely set at factory. In one embodiment, the gas in a sealed reservoir 4 is disposed at a pressure ranging from about 2 bar or 200 kPa to about 50 bar or 5 MPa. As the reservoir 4 is squeezed, the gas, e.g., nitrogen or another non-oxidizing or non-reacting gas traverses the apertures along with droplets which together form a fine or variable mist spray. The reservoir 4 is preferably constructed from a resilient material, e.g., thin but sturdy material capable of deforming in response to squeezing forces applied by a user's hand but capable of returning to the shape prior to the squeezing forces having been applied when such forces are no longer present. The reservoir 4 is preferably configured in the shape of a bulb for holding sufficient fluid to be dispensed in a compact manner. The bulbus shape further facilitates grasping of the dispenser as a user's hand can more readily feel the part that needs to be squeezed to dispense the contents of the dispenser. The liquid that traverses the screen 38 and exits as fine mist or spray at particle sizes ranging from about 0.03 mm to about 0.12 mm and at speed of from about 10 mm/s to about 100 mm/s, more preferably at about 40 mm/s.

FIG. 8 is a rear view of the embodiment shown in FIG. 1 with the cap of the ear or eye nozzle removed, i.e., the nozzle labelled with indicia “EAR/EYE” 16. Here, a slit 40 is disposed at or near the tip of neck 12 for dispensing drops. This slit 40 is sufficiently large such that when the reservoir 4 is squeezed, one or more drops exit the slit 40. A cut 60 disposed on each side of the slit 40 facilitates the deformation of the slit which together result in opening of the slit 40 to allow one or more drops to penetrate and form upon exiting the slit 40. Before the contents of the reservoir can be dispensed, one of the caps 6, 10 can be removed by securing the dispenser by its reservoir 4 with one hand and securing the cap of the intended nozzle with the other hand before the cap is twisted against its corresponding neck such that the cap is permanently separated from the neck to expose its nozzle. The cap may alternatively be pulled away from its neck by force, severing the cap from the neck to expose its nozzle. In one embodiment, the cap is configured to be a single-use barrier to isolate the contents of the reservoir from the ambient environment without any possibility of plugging its corresponding nozzle. This prevents the cap to be replaced and the dispenser reused. FIG. 9 is a diagram depicting a manner in which the nasal nozzle can be used. The neck 8 enables a user to insert its nozzle in a nostril, thereby extending the reach of the nozzle while the base of the neck is terminated by shoulders flanking the neck 8. These shoulders limit the insertion of the nozzle to only the length of the neck, preventing injury from being imparted on a nostril 48 of a user's nose 58. Note in FIG. 9 that the base of the neck 8 is sufficiently large to limit its insertion into a nostril 48 or make it sufficiently difficult to insert into the nostril beyond just the neck 8 as at least one of the shoulders is now prevented from entering the nostril 48 while allowing the tip of the neck 8 sufficient reach into the nostril 48. In use, the dispenser is disposed such that the tip of the neck 8 is disposed within the nostril 48 so that a fine mist or spray 52 can be placed close to the location at which the spray 52 is needed and the dispenser is squeezed at its reservoir 4 by means of a thumb 54 and an index finger 56 of a hand 44. A user may self-administer the dispensation of the contents of the reservoir or the dispensation of the contents may be aided or performed by another.

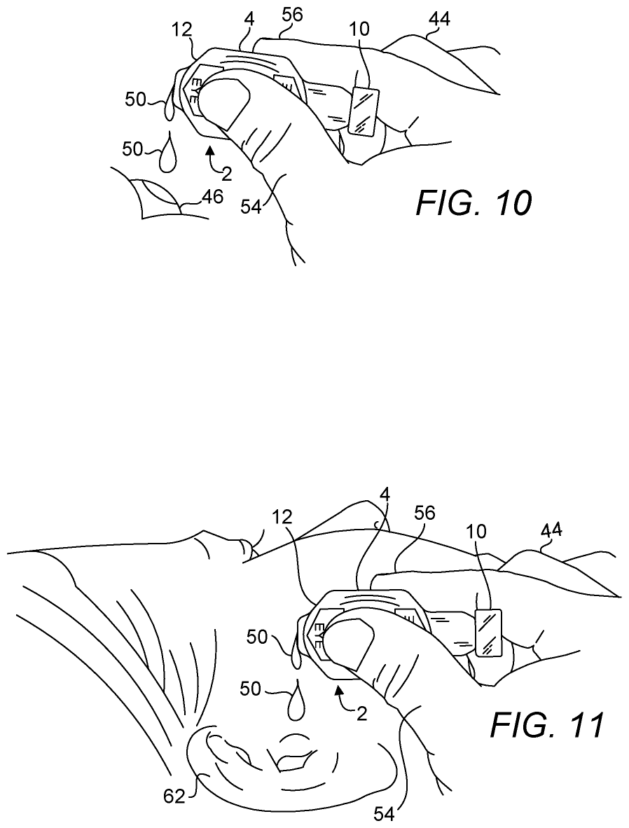

FIG. 10 is a diagram depicting a manner in which the ear or eye nozzle can be used on a user's eye. Here, it is demonstrated that, if the dispenser is used as an eye drop, upon removing the cap at the tip of neck 12, the dispenser is disposed such that one or more drops 50 can be dispensed into the eye 46 of a user by gravity. FIG. 11 is a diagram depicting a manner in which the ear or eye nozzle can be used on a user's ear.

Similar to the usage disclosed in FIG. 10, the dispenser can be used for dispensing drops 50 into the ear canal by gravity or other gravitational mechanism or methodology..

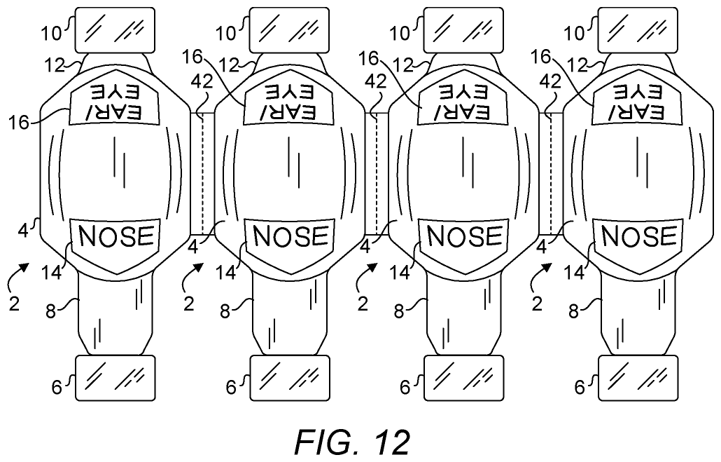

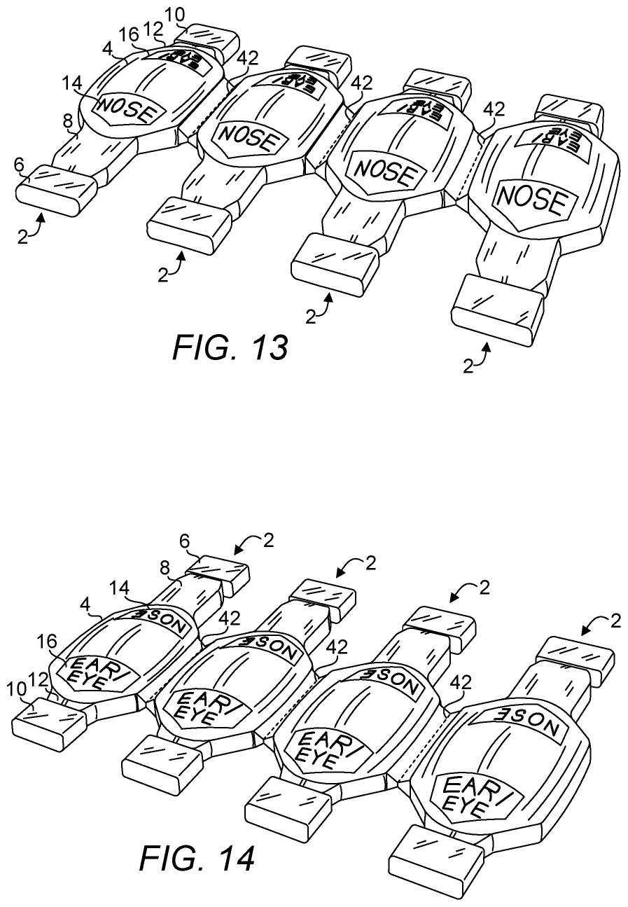

FIG. 12 is a top view of a plurality of dispensers supplied as a single group of dispensers 2. FIG. 13 is a top front perspective view thereof. FIG. 14 is a bottom rear perspective view thereof. Here, a minimum of two with an average gang of four dispensers 2 are connected together to ease handling of the dispensers 2 especially when they are not transported in a container or box together. Dispensers 2 are connected together via thin and weak joints 42 that are formed on their lengthwise sides.

Although the present dispenser has been shown to be useful for use with a fluid, the multiple delivery options of the present dispenser may be used for dispensing a substance in the form of powder or gel, provided that suitable nozzles are used.

The detailed description refers to the accompanying drawings that show, by way of illustration, specific aspects and embodiments in which the present disclosed embodiments may be practiced. These embodiments are described in sufficient detail to enable those skilled in the art to practice aspects of the present invention. Other embodiments may be utilized, and changes may be made without departing from the scope of the disclosed embodiments. The various embodiments can be combined with one or more other embodiments to form new embodiments. The detailed description is, therefore, not to be taken in a limiting sense, and the scope of the present invention is defined only by the appended claims, with the full scope of equivalents to which they may be entitled. It will be appreciated by those of ordinary skill in the art that any arrangement that is calculated to achieve the same purpose may be substituted for the specific embodiments shown. This application is intended to cover any adaptations or variations of embodiments of the present invention. It is to be understood that the above description is intended to be illustrative, and not restrictive, and that the phraseology or terminology employed herein is for the purpose of description and not of limitation. Combinations of the above embodiments and other embodiments will be apparent to those of skill in the art upon studying the above description. The scope of the present disclosed embodiments includes any other applications in which embodiments of the above structures and fabrication methods are used. The scope of the embodiments should be determined with reference to the appended claims, along with the full scope of equivalents to which such claims are entitled.

What is claimed herein is:

1. A dispenser comprising:
(a) a reservoir configured for holding a fluid; and
(b) more than one nozzle fluidly connected to said reservoir, wherein one of said more than one nozzle is a nozzle configured for dispensing one or more drops of the fluid and another one of said more than one nozzle is a nozzle configured for dispensing a spray,
wherein said reservoir is configured to be squeezable to force the fluid to at least one of said more than one nozzle to be dispensed.

2. The dispenser of claim 1, further comprising a neck disposed on one of said more than one nozzle and a shoulder disposed on a base of said neck, said neck is configured to extend the reach of the more than one nozzle into a nostril and the shoulder is configured to limit insertion of the one of the more than one nozzle to prevent injury from being imparted on the nostril.

3. The dispenser of claim 2, at least a portion of said neck comprises a weak wall configured to be breakable from a twist of a cap of said neck against said neck such that the fluid can be dispensed through said one of said more than one nozzle.

4. The dispenser of claim 2, wherein said neck is disposed at a first size at said cap and a second size away from said cap, said second size is greater than said first size and said neck is configured to taper from said first size to said second size at a taper such that said taper causes said one of said more than one nozzle to be centered while said one of said more than one nozzle is seated against the nostril.

5. The dispenser of claim 1, wherein said more than one nozzle comprises a drop dispenser.

6. The dispenser of claim 1, wherein said more than one nozzle comprises a spray dispenser.

7. The dispenser of claim 1, further comprising a breakable member having two sides, one of said two sides is configured to be connected to said dispenser and the other one of said two sides is connected to another one of such dispensers.

8. The dispenser of claim 1, wherein said one of said more than one nozzle comprises a screen to cause the fluid to exit said one of said more than one nozzle in a fine or variable mist form.

9. The dispenser of claim 1, wherein said more than one nozzle comprises two nozzles.

10. A dispenser comprising:
(a) a reservoir configured for holding a fluid;
(b) more than one nozzle fluidly connected to said reservoir; and
(c) a cap configured for sealing each said more than one nozzle such that said fluid is selectively confined in said reservoir, isolated from an ambient environment,
wherein said reservoir is configured to be squeezable to force the fluid to at least one of said more than one nozzle to be dispensed.

11. The dispenser of claim 10, further comprising a neck disposed on one of said more than one nozzle and a shoulder disposed on a base of said neck, said neck is configured to extend the reach of the more than one nozzle into a nostril and the shoulder is configured to limit insertion of the one of the more than one nozzle to prevent injury from being imparted on the nostril.

12. The dispenser of claim 11, at least a portion of said neck comprises a weak wall configured to be breakable from a twist of said cap against said neck such that the fluid can be dispensed through said one of said more than one nozzle.

13. The dispenser of claim 11, wherein said neck is disposed at a first size at said cap and a second size away from said cap, said second size is greater than said first size and said neck is configured to taper from said first size to said second size at a taper such that said taper causes said one of said more than one nozzle to be centered while said one of said more than one nozzle is seated against the nostril.

14. The dispenser of claim 10, wherein said more than one nozzle comprises a drop dispenser.

15. The dispenser of claim 10, wherein said more than one nozzle comprises a spray dispenser.

16. The dispenser of claim 10, further comprising a breakable member having two sides, one of said two sides is configured to be connected to said dispenser and the other one of said two sides is connected to another one of such dispensers.

17. The dispenser of claim 10, wherein said one of said more than one nozzle comprises a screen to cause the fluid to exit said one of said more than one nozzle in a fine or variable mist form.

18. The dispenser of claim 10, wherein said more than one nozzle comprises two nozzles.
